(19) United States
(12) Patent Application Publication
(10) Pub. No.: US 2014/0163488 A1
(43) Pub. Date: Jun. 12, 2014

(54) Pinch Clip

(71) Applicants: Michael J. Vaillancourt, Chester, NY (US); Marshall Kerr, Carlsbad, CA (US)

(72) Inventors: Michael J. Vaillancourt, Chester, NY (US); Marshall Kerr, Carlsbad, CA (US)

(21) Appl. No.: 13/709,589

(22) Filed: Dec. 10, 2012

(51) Int. Cl. F16B 2/22; A61M 1/00

(52) U.S. Cl. CPC F16B 2/22; A61M 1/0031
USPC 604/319; 24/545

(57) ABSTRACT

The pinch clip has a flange with a recess on the underside and a resiliently biased leg for moving from an open position spaced from a tubing and the flange to a closed position abutting and closing the tubing to a flow of liquid therethrough. The leg has a lip at the end for disposition in the recess of the flange to lock the leg in the closed position.

This application relates to a pinch clip. More particularly, this invention relates to a pinch clip for closing medical tubing. Still more particularly, this invention relates to a pinch clip for closing a tubing extending from a vacuum bottle for pleural effusion.

As is known, various types of pinch clips have been used for closing tubing employed with medical devices, such as described in U.S. Pat. Nos. 5,474,572; 4,453,295; 3,766,925 and 3,713,622 as well as for closing plastic bags, as described in U.S. Pat. Nos. 5,109,576 and 4,866,818 and for clamping electrical cables, as described in U.S. Pat. No. 4,623,102. Typically, these types of pinch clips have a main body and a resilient leg that is able to be pivoted from an open position relative to the body to a closed position relative to the body wherein the end of the leg engages with a protuberance on the base.

In the case of the pinch clips used for closing a tubing of a medical device, the closing leg of the pinch clip has been pressed against the tubing in order to collapse or squeeze the tubing from a circular shape to a flattened shape sufficient to close the lumen of the tubing to a flow of liquid, such as body fluids, IV fluids, blood and the like. Because of the inherent resiliency of the tubing, the leg of the pinch clip is biased outwardly from the base when in use. As a result, the engagement of the leg with the base can be compromised to such an extent that the leg pops away from the base and the tubing becomes open to the flow of fluids.

As is also known, vacuum bottles have been used in the treatment of pleural effusion. As described in US Patent Application Publication 2011/0022012, a vacuum bottles can be connected via a catheter to a chest tube implanted in a patient in order to draw fluid from the chest of a patient. In order to control the use of the vacuum bottle, a check valve has been placed between the catheter and the chest tube. Opening of the check valve allows the negative pressure in the vacuum bottle to draw fluid from the chest tube while closing of the check valve stops the flow.

It is an object of the invention to provide an improved pinch clip that provides a secure closure upon being moved into a closed position.

It is another object of the invention to reduce the risk of a closed tubing becoming opened due to a pinch clip becoming inadvertently opened.

It is another object of the invention to provide a pinch clip for closing a catheter extending from a vacuum bottle for pleural effusion procedures.

It is another object of the invention to provide a pinch clip with a locking feature.

Briefly, the invention is directed to a pinch clip comprised of a base having an upstanding abutment thereon and a finger-gripping surface at one end thereof and a leg that extends from the base in cantilevered manner and is resiliently movable relative to the base to move between an open position and a closed position. The leg also has an abutment facing the abutment on the base for clamping a tubing passing therebetween when the leg is in the closed position.

In one embodiment, the leg has an aperture for passage of a tubing therethrough longitudinally of base and a wall extends vertically from the base and has an aperture for passage of the tubing therethrough longitudinally of the base. In addition, a tab extends from the wall and forms a second finger gripping surface opposite and spaced from finger-gripping surface on the base. Pinching of the two finger-gripping surface towards each other upon the application of manual pressure causes the resilient leg to move from the closed position to the open position.

In accordance with the invention, a flange extends from the wall on a side opposite the tab and has a recess in an underside while the leg has a lip at the end that extends outwardly from the leg to be disposed in the recess of the flange in the closed position of the leg and to be spaced from the flange in the open position of the leg.

When in the closed position, the leg of the pinch clip is securely locked in place by the engagement of the lip in the recess of the flange. In this respect, the force that biases the leg from the closed position to the opened position biases the lip to move further into the recess such that slippage of the end of the leg from the flange of the base is counter-acted.

When the pinch clip is to be intentionally opened, manual gripping and squeezing together of the two finger-gripping surfaces causes the wall of the base to flex away from the leg and the flange to be pivoted away from the lip on the leg. As the lip moves out of the recess in the underside of the flange, the leg returns to the opened position spaced from the flange.

The pinch clip is particularly useful in closing a tubing or catheter extending from a vacuum bottle. In this respect, the vacuum bottle has an indicator for indicating the existence of a vacuum within the bottle, an inlet port and a rubber tube extending from the inlet port and to which a tubing is secured for effusion of liquids into the bottle during a pleural effusion procedure.

The pinch clip is employed on the rubber tube by being slid along the tubing to a point of use on the rubber tube with the clip in the opened position. The clip is then closed by moving the resilient leg into engagement with the underside of the flange of the base. At this time, the two abutments of the clip squeeze the rubber tube into a closed state.

These and other objects and advantages of the invention will become more apparent from the following detailed description taken in conjunction with the accompanying drawings wherein:

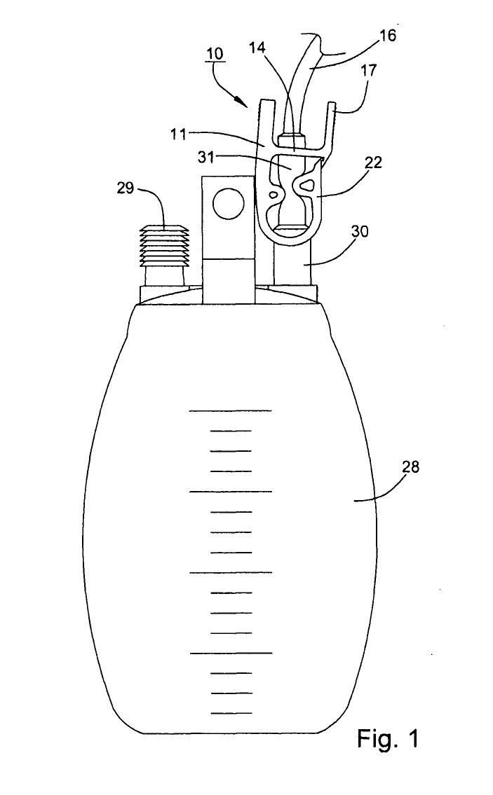

FIG. 1 illustrates a side view of a vacuum bottle employing a pinch clip in accordance with the invention;

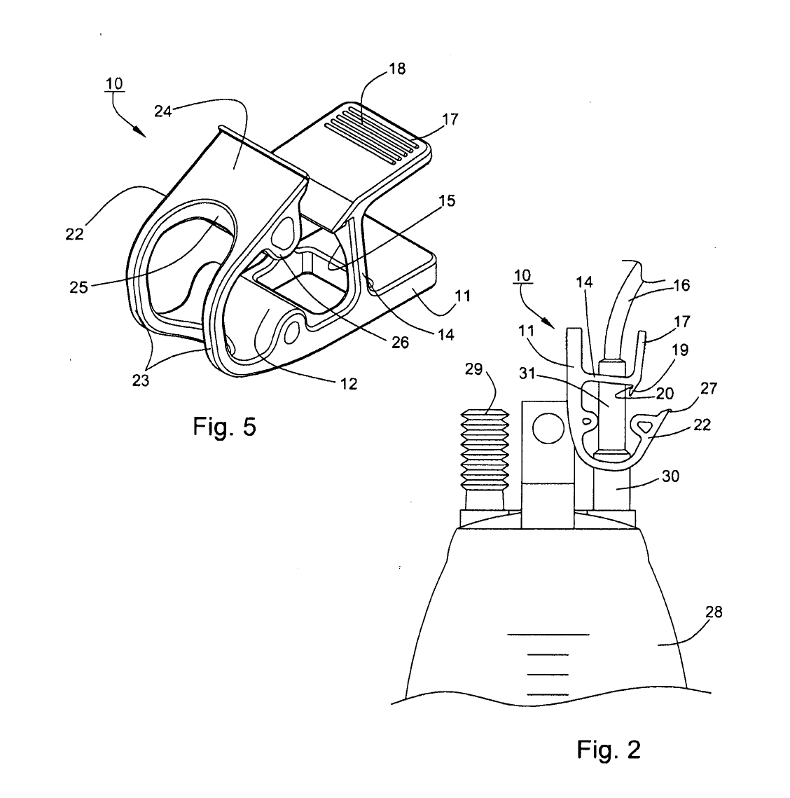

FIG. 2 illustrates a view similar to FIG. 1 with the pinch clip in an opened position;

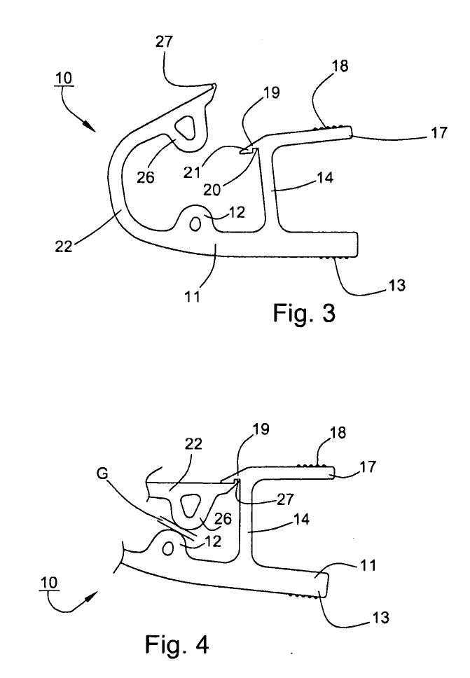

FIG. 3 illustrates a side view of the pinch clip of FIG. 1 in an opened position;

FIG. 4 illustrates a partial side view of the pinch clip of FIG. 1 in a closed position; and

FIG. 5 illustrates a perspective view of the pinch clip of FIG. 1.

Referring to FIG. 3, the pinch clip 10 is made as a unitary body of plastic material and includes a base 11 having an upstanding abutment 12 thereon, a finger-gripping surface 13 at one end and a wall 14 that extends upwardly, as viewed, between the abutment 12 and finger-gripping surface 13.

The abutment 12 has a rounded outer surface for purposes as described below and is transverse to the base 11 and the finger-gripping surface 13 has a roughened surface formed, for example, by ribs or a series of protuberances.

Referring to FIG. 5, the wall 14 is of a thickness to be resiliently flexible relative to the base 11 that is of greater thickness. In addition, the wall 14 has a central aperture 15 for passage of a tubing 16 (see FIG. 1) therethrough longitudinally of the base 11.

The pinch clip 10 also has a tab 17 that extends from the wall 14 in a perpendicular manner and that forms a second finger gripping surface 18 opposite and spaced from the finger-gripping surface 13 of the base 11. The finger-gripping surface 18 also has a roughened surface formed, for example, by ribs or a series of protuberances.

The pinch clip 10 also has a flange 19 extending from the wall 14 on a side opposite the tab 17. As illustrated in FIG. 3, the flange 19 has a recess 20 in the underside and the topside has a sloped surface 21.

The pinch clip 10 also has a leg 22 that extends from the base 11 in cantilevered manner and that is bent back on itself to form a U-shaped projection from the base 11. The leg 22 is resiliently movable relative to the base 11 to move between an open position as shown in FIG. 3 and a closed position as shown in FIG. 4.

Referring to FIG. 5, the leg 22 is of skeletal construction with a pair of limbs 23 that extend from the base 11 and a flat plate-like component 24 that extends from the ends of the limbs 23. The limbs 23 thus define an aperture 25 for the passage of the tubing 16 (see FIG. 1) therethrough longitudinally of the base 11 and provide for a spring-like effect to move the leg 22 from the closed position of FIG. 4 to the opened position of FIG. 3.

The leg 22 also has an abutment 26 on the flat plate-like component 24 that has a rounded outer surface facing and parallel to the abutment 12 on the base 11 for clamping a tubing 16 passing through the clip 10 in the closed position. When brought towards each other, the abutments 12, 26 form a gap G (see FIG. 4) of 0.120 inches maximum for clamping purposes.

The leg 22 also has a lip 27 extending outwardly from the end of the leg 22 in a direction transverse to the flange 19 when in the closed position of FIG. 4. As shown, the lip 27 is sized to be disposed in the recess 20 of the flange 19 in the closed position of the leg 22. When the leg 22 is in the opened position, as shown in FIG. 3, the lip 27 is spaced from the flange 19 and is disposed above the plane of the flange 19

The tab 17 is integral with the flange 19 for flexing with the wall 14 between a first position maintaining the lip 27 in the recess 20 of the flange 19 and a second position to move the flange 19 from the lip 27 to allow the leg 22 to resiliently move from the closed position to the open position.

Referring to FIG. 1, the pinch clip 10 is particularly useful for closing off the tubing 16 extending from a vacuum bottle 28 used for pleural effusion.

As illustrated, the vacuum bottle 28 is of conventional structure having an indicator 29 for indicating the existence of a vacuum within the bottle 28, an inlet port 30 to the interior of the bottle 28 and a rubber tube 31 that extends from within the port 30 and to which the tubing 16 is secured for the effusion of liquids into the bottle.

In use, the pinch clip 10 in an opened position is slid over the end of the tubing 16 and moved into position over the rubber tube 31 and adjacent the inlet port 30 of the bottle.

Next, the two finger-gripping surfaces 13, 18 are squeezed towards each other causing the wall 14 to bend in a direction away from the leg 22, to the right as viewed, while the leg 22 of the clip 10 is manually depressed from the position as shown in FIG. 3 to bring the lip 27 into alignment with the recess 20 in the flange 19. The two finger-gripping surfaces 13, 18 are then released to allow the wall 14 to flex back in the direction towards the leg 22, to the left as viewed in FIG. 3, and to allow the leg 22 to flex upwardly, as viewed, to cause the lip 27 to engage in the recess 20 as indicated in FIG. 4 thereby locking the leg 22 in the closed position of FIG. 4. During this time, the abutment 26 on the leg 22 is moved toward the abutment 12 on the base 11 to squeeze the rubber tube 31 into a flattened shape closing off the lumen of the tube 31. In this respect, the gap G between the abutments 12, 26 is sufficient to close the lumen of the tube 31 to a flow of fluid.

Referring to FIG. 2, in order to operate the vacuum bottle 28 to draw fluids from a chest cavity of a patient, the two finger-gripping surfaces 13, 18 are again squeezed towards each other causing the wall 14 to bend away from the leg 22 and allowing the lip 27 to slide out of the recess 20 and along the underside of the flange 19 into the opened position under the inherent bias of the limbs 23. The rubber tube 31 is thus allowed to open to the flow of fluids into the bottle 28. Upon expiration of the vacuum within the bottle 28, the indicator 29 responds by moving outwardly as is conventional.

The invention thus provides an improved pinch clip that provides a secure closure upon being moved into a closed position and that reduces the risk of a closed tubing becoming opened due to a pinch clip becoming inadvertently opened.

The invention further provides a pinch clip with a locking feature and one that is particularly useful for closing a tubing or catheter extending from a vacuum bottle for pleural effusion procedures.

1. A pinch clip comprising
a base having a first upstanding abutment thereon and a first finger-gripping surface at one end thereof;
a wall extending from said base and having an aperture for passage of a tubing therethrough longitudinally of said base;
a tab extending from s wall and forming a second finger gripping surface opposite and spaced from said first finger-gripping surface;
a flange extending from said wall on a side opposite said tab, said flange having a recess therein;
a leg extending from said base in cantilevered manner and being resiliently movable relative to said base to move between an open position and a closed position, said leg having an aperture for passage of a tubing therethrough longitudinally of said base an a second abutment thereon facing said first abutment for clamping a tubing passing through said aperture therebetween in said closed position; and
a lip extending from said leg and being disposed in said recess of said flange in said closed position of said leg and spaced from said flange in said open position of said leg.

2. A pinch clip as set forth in claim 1 made of a one-piece unitary body of plastic material.

3. A pinch clip as set forth in claim 1 wherein said first abutment is transverse to said base and said second abutment is parallel to said first abutment.

4. A pinch clip as set forth in claim 3 wherein said first abutment has a rounded surface facing said second abutment and said second abutment has a rounded surface facing said first abutment.

5. A pinch clip as set forth in claim 1 wherein said lip is disposed above the plane of said flange and spaced from said flange in said open position of said leg.

6. A pinch clip as set forth in claim 5 wherein said lip extends outwardly of said leg in a direction transverse to said flange in said closed position of said leg.

7. In combination
a vacuum bottle having an indicator for indicating the existence of a vacuum within said bottle, an inlet port and a rubber tube extending from said inlet port;
a tubing secured to said rubber tube for effusion of liquids into said bottle; and
a pinch clip for selectively closing said rubber tube to a flow of liquid therethrough. said pinch clip having a base with a first finger-gripping surface at one end thereof a wall extending from said base and having an aperture for passage of a tubing therethrough longitudinally of said base, a tab extending from said wall and forming a second finger gripping surface opposite and spaced from said first finger-gripping surface; a flange extending from said wall thereon with a recess therein, a resiliently biased leg extending from said base for moving from an open position spaced from said tubing and said flange to a closed position abutting and closing said rubber tube to a flow of liquid therethrough, and a lip extending from said leg for disposition in said recess of said flange in said closed position of said leg.

8. The combination as set forth in claim 7 wherein said lip extends outwardly of said leg in a direction transverse to said flange in said closed position of said leg.

9. The combination as set forth in claim 7 wherein said leg of said pinch clip has an aperture for passage of said tubing and said rubber tube longitudinally therethrough.

10. The combination as set forth in claim 9 wherein said leg has an abutment transverse to said tubing for abutting and closing said rubber tube to a flow of liquid in said closed position.

11. (canceled)

12. A pinch clip comprising
a base having a first upstanding abutment thereon, a first finger-gripping surface at one end thereof and a wall extending from said base between said abutment and said finger-gripping surface, said wall having an aperture for passage of a tubing therethrough longitudinally of said base and being resiliently flexible relative to said base and being of a thickness less than the thickness of said base;
a tab extending from said wall and forming a second finger gripping surface opposite and spaced from said first finger-gripping surface;
a flange extending from said wall on a side opposite said tab, said flange having a recess therein;
a leg extending from said base in cantilevered manner and being resiliently movable relative to said base to move between an open position and a closed position, said leg having aperture for passage of a tubing therethrough longitudinally of said base and a second abutment thereon facing said first abutment for clamping a tubing passing through said aperture therebetween in said closed position; and
a lip extending from said leg an being disposed in said recess of said flange in said closed position of said leg and spaced from said flange in said open position of said leg.

13. A pinch clip as set forth in claim 12 wherein said first finger-gripping surface and said tab are movable toward each other to effect bending of said wall in a direction away form said leg.

14. A pinch clip as set forth in claim 12 wherein said wall is perpendicular to said base and said first finger-gripping surface.
